(19) United States
(12) Patent Application Publication
(10) Pub. No.: US 2022/0313803 A1
(43) Pub. Date: Oct. 6, 2022

(54) NOVEL NEOANTIGENS AND CANCER IMMUNOTHERAPY USING SAME

(71) Applicant: BRIGHTPATH BIOTHERAPEUTICS CO., LTD., Kawasaki-shi, Kanagawa (JP)

(72) Inventors: Tetsuro SASADA, Yokohama-shi, Kanagawa (JP); Junya OHTAKE, Yokohama-shi, Kanagawa (JP); Susumu IIZUMI, Kawasaki-shi, Kanagawa (JP); Norihiro NAKAMURA, Kawasaki-shi, Kanagawa (JP); Fumiko ISODA, Kawasaki-shi, Kanagawa (JP); Kazuhiko ODAKA, Tokyo (JP)

(21) Appl. No.: 17/420,963

(22) PCT Filed: Jan. 6, 2020
(86) PCT No.: PCT/JP2020/000018

§ 371 Date: Feb. 22, 2022

(30) Foreign Application Priority Data
Jan. 7, 2019 (JP) 2019-000903

(51) Int. Cl. A61K 39/00; A61P 35/00; A61K 35/17

(52) U.S. Cl. CPC A61K 39/0011; A61P 35/00; A61K 35/17; A61K 2039/572; A61K 2039/5154

(57) ABSTRACT

Problem to be solved: An object of the present invention is to obtain medicinal effect that could not be obtained by conventional peptide vaccines that activate and proliferate a CD8-positive CTL by administering a Class II epitope as a peptide vaccine. Solution: The inventors of the present invention have found, as a result of diligent examination on the aforementioned problems, that these problems can be solved by acquiring a peptide having a partial amino acid sequence containing a mutated amino acid of a neoantigen expressed in cancer cells and being an epitope presented by a Class II molecule.

### CROSS-REFERENCE

This application is a National Stage Entry of PCT/JP2020/000018, filed Jan. 6, 2020, which claims the benefit of Japanese Provisional Application No. 2019-000903, filed Jan. 7, 2019, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is tbd_SEQUENCE_LISTING.txt. The text file is 11 KB, was created on Jul. 3, 2021, and is being submitted electronically via EFS-Web.

### TECHNICAL FIELD

The present invention relates to neoantigens for treatment or prevention of cancer. More specifically, the present invention relates to neoantigens derived from tumor-specific antigens, cancer driver mutated proteins, cancer passenger mutated proteins, etc., which are specifically generated in cancer patients. More specifically, the present invention relates to neoantigens derived from driver mutations that are frequently shared by cancer patients.

### BACKGROUND ART

Cancer vaccine therapy is a cancer treatment method that aims to prevent or treat cancer by activating and proliferating a specific immune response in the patient's body against cancer antigens expressed on cancer cells, in which an antigen formed of a cancer antigen protein or a peptide that is a part thereof or a gene (DNA or RNA) encoding such an antigen is administered as a vaccine. Since the 1990s, the research on many cancer antigens and development of vaccines targeting them have been progressed, but a therapeutic effect has not been proven in most clinical trials.

Many of the cancer vaccine therapies examined so far have targeted to “tumor-related antigens” derived from wild-type self-antigens without genetic mutations, but since these antigens are self-antigens, the specific T cells that are highly responsive to these antigens, disappeared from the body due to the mechanism of immune tolerance, and sufficient immune response was not obtained, which has been conjectured to be one of the causes of the failure of vaccine development so far.

On the other hand, the novel antigen, neoantigen, generated by a genetic mutation is recognized as “non-self” by the living body because it does not naturally exist in vivo, and, therefore, the neoantigen is considered to efficiently induce the immune reaction. In particular, among neoantigens, antigens derived from driver mutations that are frequently shared by cancer patients are promising as candidates for cancer therapeutic agents. In fact, a plurality of neoantigens derived from driver mutations has been identified, and attempts have been made to develop peptide vaccines targeting these (Non-Patent Reference 1, clinical trial No. NCT02454634). Most of the peptide vaccines currently being developed are composed of 12-mer or less amino acids and are presented on an HLA Class I molecule of antigen-presenting cells (Class I epitope), and activate and proliferate CD8-positive cytotoxic T cells (CTL). The CD8-positive CTL plays a major role in tumor immunity and attacks cancer cells that present the epitope on HLA molecules.

On the other hand, in recent years, the importance of CD4-positive helper T cells in the anti-tumor immune response has been frequently reported. It has been reported that the CD4-positive helper T cell has a direct antitumor ability in addition to functions in dendritic cell licensing and abilities in maintenance and activation of the CD8-positive CTL (Non-Patent Reference 2).

The CD4-positive T cell is activated and proliferated by 13 to 25 mer known to be peptide (Class II epitope) presented on the HLA Class II molecule of antigen-presenting cells. In contrast, the Class I epitope currently being developed can activate and proliferate the CD8-positive CTL, but not the CD4-positive helper T cell. So far, there exist literatures reporting that a peptide vaccine frequently expressed in tumor tissues, containing a major driver mutation, can activate and proliferate CD4-positive T cells (i.e., a Class II epitope) (e.g., IDH1-R132H that is a driver mutation in brain tumors (Non-Patent Reference 3) and P53-R248W and KRAS-G12V that are prominent driver mutations (Non-Patent Reference 4)), however, these are just examples of immunogenicity and medicinal effect in the experimental system using mice.

### LIST OF PRIOR ART REFERENCES

### Non-Patent Reference Literature

Non-Patent Reference 1: Tran et al., Science 350, 1387-1390 (2015)
Non-Patent Reference 2: Tran et al., Science 344, 641-645 (2014)
Non-Patent Reference 3: Schumacher et al., Nature 512, 324-327 (2014)
Non-Patent Reference 4: Quandt et al., OncoImmunity, 7, e1500671, (2018) (https://doi.org/10.1080/2162402X.2018.1500671)

### SUMMARY OF INVENTION

### Technical Problem

The present invention relates to neoantigens for treatment or prevention of cancer. More specifically, the present invention relates to neoantigens derived from tumor-specific antigens, cancer driver mutated proteins, cancer passenger mutated proteins, etc., which are specifically produced in cancer patients. More specifically, the present invention relates to neoantigens derived from driver mutations that are frequently shared by cancer patients.

An object of the present invention is to obtain medicinal effect that could not be obtained by conventional peptide vaccines that activate and proliferate a CD8-positive CTL by administering a Class II epitope as a peptide vaccine. Furthermore, it is also an object to provide a therapeutic effect by identifying, cloning, and proliferating CD4-positive helper T cells induced by the peptide, and transferring them to a patient. Further, an object of the present invention is to create TCR gene-modified T cells (TCR-T) by identifying a T cell receptor (TCR) gene sequence for the antigens from the CD4-positive T cells and introducing the genes into the T cells, and to use them as a therapeutic agent. However, there exist no reports of a peptide vaccine containing driver mutations (for example, mutations occurring in the PIK3CA and C-Kit genes) in cancer types with a large number of patients, such as colorectal cancer and breast cancer.

### Solution to Problem

The inventors of the present invention have found, as a result of diligent examination on the aforementioned problems, that these problems can be solved by acquiring a peptide having a partial amino acid sequence containing a mutated amino acid of a neoantigen expressed in cancer cells, which is an epitope presented by a Class II molecule.

More specifically, the present application provides the following embodiments in order to solve these problems:

[1] A peptide having a partial amino acid sequence comprising a mutated amino acid of a neoantigen, which is an epitope presented by an HLA Class II molecule;
[2] The peptide according to [1], wherein the peptide activates and proliferates a CD4-positive helper T cell;
[3] The peptide according to [1] or [2], wherein the peptide is derived from an amino acid sequence comprising a tumor-specific antigen, a cancer driver mutated protein, or a cancer passenger mutated protein;
[4] The peptide according to any one of [1] to [3], wherein the peptide has 9 to 27 amino acids length;
[5] The peptide according to any one of [1] to [4], wherein the cancer driver mutation is selected from the group consisting of PIK3CA-H1047R, C-Kit-D816V, NRAS-Q61R, KRAS-G12D, KRAS-G12R, and KRAS-G13D;
[6] The peptide according to any one of [1] to [5], wherein the peptide further has antigenicity as an HLA Class I-restricted epitope (having an ability to activate and proliferate a CD8-positive antigen-specific T cell);
[7] The peptide according to any one of [1] to [6], wherein the peptide comprises a partial sequence of an amino acid sequence selected from SEQ ID NO: 1 to SEQ ID NO: 10;
[8] The peptide according to any one of [1] to [6], wherein the peptide consists of any one of amino acid sequences selected from SEQ ID NO: 11 to SEQ ID NO: 27;
[9] A peptide vaccine against cancer which comprises the peptide according to any one of [1] to [8];
[10] The peptide vaccine according to [9], wherein the peptide vaccine activates an immune cell selected from the group consisting of a CD8-positive T cell, a CD4-positive T cell, a yoT cell, a NK cell, a NKT cell, a dendritic cell, and a macrophage;
[11] A method for activating and proliferating an antigen-specific T cell, comprising a step of contacting lymphocytes with the peptide according to any one of [1] to [8];
[12] A method for preparing an antigen-presenting cell, comprising a step of contacting a cell having an antigen-presenting ability with the peptide according to any one of [1] to [8];
[13] The method according to [12], in which contacting the cell having an antigen-presenting ability with the peptide is performed by:

a step of culturing the cell with the peptide, and binding and presenting the peptide to an HLA molecule of the cell, or

a step of introducing a vector capable of expressing the peptide into the cell to express the peptide;

[14] The method according to [12] or [13], wherein the cell having an antigen-presenting ability is a dendritic cell;
[15] A gene encoding a T cell receptor (TCR) reactive to the peptide according to any one of [1] to [8] isolated from an antigen-specific T cell clone for the peptide;
[16] The gene according to [15], wherein the gene encoding a TCR is a gene encoding a TCRcc chain or a gene encoding a TCRI3 chain, or both thereof;
[17] The gene according to [15] or [16], wherein the gene is the full length or a part of a complementarity determining region (CDR) gene;
[18] A T cell having a modified TCR gene (TCR-T cell), created by introducing the gene according to any one of [15] to [17] into a T cell.

### Advantageous Effects of Invention

All the peptides which were confirmed to have immunogenicity in the present invention function as Class II epitopes and activate and proliferate CD4-positive T cells. Moreover, the peptides of obtained from the two types of the confirmed peptides, PIK3CA-H1047R and C-Kit-D816V, also function as Class I epitopes and activate and proliferate CD8-positive T cells. So far, a peptide antigen containing each driver mutation of PIK3CA-H1047R, NRAS-Q61R, KRAS-G12D, KRAS-G12R, KRAS-G13D and C-Kit-D816V, which function as a Class II epitope, and activate and proliferate CD4-positive T cells, has not been known.

Therefore, the present invention relates to a cancer cell-derived neoantigen available for treatment or prevention of cancer. More specifically, it relates to a driver mutation-derived neoantigen that is frequently shared by cancer patients.

### BRIEF DESCRIPTION OF DRAWINGS

FIG. 1 is a diagram showing amino acid sequences each of which is a portion containing a mutated amino acid of a driver mutation-derived neoantigen peptide.

FIG. 2 is a diagram showing steps of immunogenicity evaluation of each synthesized peptide.

FIG. 3 is diagrams showing results of antigen-specific T cell activation by intracellular cytokine staining (ICS).

FIG. 4 is diagrams showing results of immunogenicity evaluation of each peptide by using PBMCs.

FIG. 5-1 FIG. 5 is diagrams showing results of confirming HLA Class II restriction by a Blocking Assay using an anti-HLA Class II antibody.

FIG. 5-2 FIG. 5 is diagrams showing results of confirming HLA Class II restriction by a Blocking Assay using an anti-HLA Class II antibody.

FIG. 5-3 FIG. 5 is diagrams showing results of confirming HLA Class II restriction by a Blocking Assay using an anti-HLA Class II antibody.

FIG. 6-1 FIG. 6 is diagrams showing results of identifying an allele of T cells of which DP/DQ/DR restriction was identified by a Blocking Assay, by specificity confirmation using an allogeneic B cell line as antigen-presenting cells (APC).

FIG. 6-2 FIG. 6 is diagrams showing each result of identifying an allele of T cells of which DP/DQ/DR restriction was identified by a Blocking Assay, by specificity confirmation using an allogeneic B cell line as APC.

FIG. 7-1 FIG. 7 is diagrams showing results of epitope mapping of neoantigen candidates. FIG. 7-1 shows results of the epitopes of the PIK3CA-H1047R-specific T cells derived from the donor No. 3.

FIG. 7-2 FIG. 7 is diagrams showing results of epitope mapping of neoantigen candidates. FIG. 7-2 shows results of the epitopes of the PIK3CA-H1047R-specific T cells derived from the donor No. 9. FIG. 7-3 FIG. 7 is diagrams showing results of epitope mapping of neoantigen candidates. FIG. 7-3 shows results of the epitopes of the C-Kit-D816V-specific T cell derived from the donor No. 10.

FIG. 8 is a diagram showing amino acid sequences of the TCR chains of the neoantigen-specific T cells (in which the CDR3 region of each chain is underlined).

FIG. 9 is a diagram showing that, when T cells which were introduced with neoantigen-specific TCR genes (TCR-T cells) are cultured together with antigen-presenting cells (APC) in the presence of the neoantigen peptide, T cells producing interferon gamma (IFNy) increase remarkably in neoantigen peptide-specific manner.

### DESCRIPTION OF EMBODIMENTS

The terms used in the present invention are defined as follows:

(a) neoantigen: an antigen containing an amino acid mutation derived from a genetic mutation occurring in a cancer cell;
(b) antigen: a protein having immunogenicity in vivo or a peptide that is a part thereof;
(c) cancer vaccine: an antigen or a gene encoding it (DNA or RNA) that is administered to a human body for the purpose of preventing or treating cancer;
(d) neoantigen vaccine: a cancer vaccine that targets a neoantigen, including those inducing cell-mediated immunity and those inducing humoral immunity;
(e) driver mutation: a genetic mutation that is generated in a cancer cell and is directly involved in canceration of the cell;
(f) passenger mutation: a genetic mutation that is generated in a cancer cell and is not directly involved in canceration of the cell;
(g) epitope: an amino acid sequence of a part of an antigen that binds to an HLA Class I or HLA Class II molecule;
(h) cancer immunotherapeutic agent: a compound, such as peptide, or cells, administered to a human body for the purpose of prevention or treatment of cancer, or a compound, such as a peptide, used for preparing cells ex vivo for prevention or treatment of cancer. The cells include T cells and dendritic cells that were stimulated by antigens to kill tumors, genetically modified T cells, etc.
(i) activation: activation in the present invention means that TCR on the surface of a T cell recognizes a peptide bound to HLA on the surface of an antigen-presenting cell (APC), then signal transduction occurs in the T cell, and release of cytotoxic granules and expression of various genes, such as for cytokines (IFNy, etc.) occur. Activation of T cells results in proliferation thereof

The inventors of the present invention have found, as a result of diligent examination on the aforementioned problems, that these problems can be solved by acquiring a peptide having a partial amino acid sequence containing a mutated amino acid of a neoantigen expressed in cancer cells, which is an epitope presented by a Class II molecule.

Peptide

In one embodiment, the present invention provides a peptide having a partial amino acid sequence containing a mutated amino acid of a neoantigen, which is an epitope presented by an HLA Class II molecule.

First, the present inventors examined as a target a protein that is characteristically expressed in cancer to obtain a peptide that can be utilized as a cancer peptide vaccine. In the present specification, as the protein characteristically expressed in cancer, the present inventors decided to examine neoantigens that are novel antigens generated by a genetic mutation in cancer cells, because the neoantigens are recognized as “non-self” by a living body and are expected to efficiently induce an immune reaction since they do not originally exist in vivo. The peptide in each of these neoantigens that can be used in the present invention is a peptide having a partial amino acid sequence of the neoantigen containing a mutated amino acid generated by the genetic mutation.

Next, the present inventors examined peptides having characteristic activities to obtain peptides having medicinal effect that could not be exhibited by the conventional cancer peptide vaccines. Most of the peptide vaccines currently being developed in the art are characterized that the peptide vaccines are presented on the HLA Class I molecule of antigen-presenting cells (Class I epitope) and activate and proliferate CD8-positive cytotoxic T cells (CTL). In search of obtaining characteristic medicinal effect different from that of the conventional cancer peptide vaccines, the present inventors searched for a peptide presented on the HLA Class II molecule (Class II epitope) from peptides having partial amino acid sequences containing a mutated amino acid of neoantigen proteins which are characteristically expressed on cancer cells. After having prepared candidate peptides, a Class II epitope can be obtained as an index of the peptide's activating a CD4-positive helper T cell or producing IFNy.

In the present specification, the target neoantigen may be any protein that is expressed in cancer cells but not in non-cancer cells, and can be selected from tumor-specific antigens, cancer driver mutated proteins, cancer passenger mutated proteins, etc. In particular, among the neoantigens, an antigen derived from driver mutation that is frequently shared by cancer patients is considered to be a promising candidate for cancer therapeutic agents, and therefore, a procedure of designing peptide sequences from known driver mutations is illustrated below as an example.

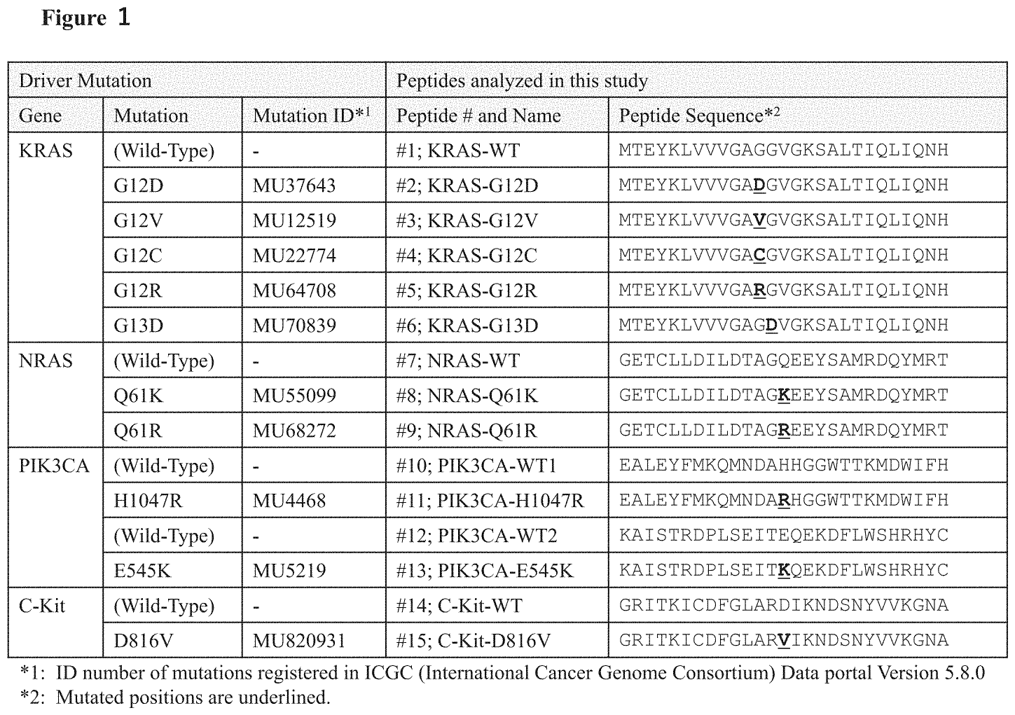

Specifically, as shown in FIG. 1, neoantigens containing the following cancer driver mutations were examined:

KRAS gene:

G12D (KRAS-G12D, mutation ID: MU37643),

G12V (KRAS-G12V, mutation ID: MU12519),

G12C (KRAS-G12C, mutation ID: MU22774),

G12R (KRAS-G12R, mutation ID: MU64708),

G13D (KRAS-G13D, mutation ID: MU70839);

NRAS gene:

Q61K (NRAS-Q61K, mutation ID: MU55099),

Q61R (NRAS-Q61R, mutation ID: MU68272);

PIK3CA gene:

H1047R (PIK3CA-H1047R, mutation ID: MU4468),

E545K (PIK3CA-E545K, mutation ID: MU5219);

C-Kit gene:

D816V (C-Kit-D816V, mutation ID: MU820931).

The sequences of the partial peptides (target sequences) containing a mutated amino acid of neoantigens derived from respective driver mutations are as follows:

TABLE 1

| | Mutation | | |
|---|---|---|---|
| Gene | name | Peptide sequence* | SEQ ID NO. |
| KRAS | KRAS-G12D | MTEYKLVVVGA [symbol] GVGKSALTIQLIQ | SEQ ID NO: 1 |
| | | NH | |
| | KRAS-G12V | MTEYKLVVVGA [symbol] GVGKSALTIQLIQ | SEQ ID NO: 2 |
| | | NH | |
| | KRAS-G12C | MTEYKLVVVGA [symbol] GVGKSALTIQLIQ | SEQ ID NO: 3 |
| | | NH | |
| | KRAS-G12R | MTEYKLVVVGA [symbol] GVGKSALTIQLIQ | SEQ ID NO: 4 |
| | | NH | |
| | KRAS-G13D | MTEYKLVVVGAG [symbol] VGKSALTIQLIQ | SEQ ID NO: 5 |
| | | NH | |
| NRAS | NRAS-Q61K | GETCLLDILDTAG [symbol] EEYSAMRDQY | SEQ ID NO: 6 |
| | | MRT | |
| | NRAS-Q61R | GETCLLDILDTAG [symbol] EEYSAMRDQY | SEQ ID NO: 7 |
| | | MRT | |
| PIK3CA | PIK3CA- | EALEYFMKQMNDA [symbol] HGGWTTKMD | SEQ ID NO: 8 |
| | H1047R | WIFH | |
| | PIK3CA-E545K | KAISTRDPLSEIT [symbol] QEKDFLWSHRHY | SEQ ID NO: 9 |
| | | C | |
| C-Kit | C-Kit-D816V | GRITKICDFGLAR [symbol] IKNDSNYVVKG | SEQ ID NO: 10 |
| | | NA | |

*The mutated amino acid is underlined in the peptide.

Based on the amino acid sequences of neoantigens thus obtained, epitopes presented by HLA Class II molecules (i.e., Class II epitopes) can be designed and prepared from partial peptides containing a mutated amino acid of neoantigens by activation of CD4-positive helper T cells as an index. Specifically, for example, among the full-length amino acid sequence of the neoantigen derived from the aforementioned driver mutated protein, peptides or partial peptides thereof having amino acid sequences of SEQ ID NO: 1 to SEQ ID NO: 10 containing a mutated amino acid can be selected as target peptides. In the present invention, a peptide having a target amino acid sequence may be a part of the sequence of any peptide containing a mutated amino acid as described above. For example, a peptide having a length of 9 to 27 amino acids containing a mutated amino acid can be used, and in a preferred embodiment, a peptide having a length of 13 to 27 amino acids containing a mutated amino acid is used.

As a result of searching for peptides derived from neoantigens obtained from cancer driver mutant proteins by the ability to activate CD4-positive helper T cells as an index, it was revealed that the peptides derived from PIK3CA-H1047R (SEQ ID NO: 8), C-Kit-D816V (SEQ ID NO: 10), NRAS-Q61R (SEQ ID NO: 7), KRAS-G12D (SEQ ID NO: 1), KRAS-G12R (SEQ ID NO: 4), and KRAS-G13D (SEQ ID NO: 5), have particularly strong ability to activate CD4-positive helper T cells. Therefore, the present invention can use the peptides having such sequences as neoantigens. The present invention can use more preferably the peptides derived from PIK3CA-H1047R (SEQ ID NO: 8), C-Kit-D816V (SEQ ID NO: 10), NRAS-Q61R (SEQ ID NO: 7), and KRAS-G13D (SEQ ID NO: 5). Based on this result, in the present specification, the neoantigen-derived peptides obtained from these cancer driver mutations can be examined as target candidate peptides for further analysis.

Whether or not the peptide activates CD4-positive helper T cells can be investigated, for example, by stimulating peripheral blood mononuclear cells (PBMCs) with the peptide and staining the peptide-stimulated PBMCs with an anti-CD4 antibody and an anti-IFNy antibody.

The peptide of the present invention is an epitope presented by an HLA Class II molecule (i.e., Class II epitope) as described above, and may also have antigenicity as an HLA Class I-restricted epitope. Such a peptide having antigenicity as the HLA Class I-restricted epitope can be acquired by an ability to activate a CD8-positive CTL as an index. Such a peptide that is both the Class II epitope and the HLA Class I-restricted epitope can more efficiently generate a cell-mediated immune reaction against cancer in vivo and can generate various immune reactions in a simultaneous manner.

Alternatively, the peptide can more effectively provide an action as a cancer peptide vaccine or a cancer immunotherapy inducer, such as more efficiently activating the target CD8-positive CTL ex vivo.

As a preferred embodiment of the present invention, shorter length peptides can be searched. Specifically, the present invention identifies shorter length peptides having an ability to activate the T cells by preparing peptides each of which have a partial sequence of the peptide derived from the aforementioned preferable neoantigen and also contain a mutated amino acid, and selecting by the activation of T cells specific to the neoantigen as an index. As a result of examining an ability of peptides derived from, for example, PIK3CA-H1047R (SEQ ID NO: 8) and C-Kit-D816V (SEQ ID NO: 10) to activate T cells specific to each neoantigen, it is revealed that the following peptides could be obtained as peptides having target actions. The present specification only describes the results relating to PIK3CA-H1047R (SEQ ID NO: 8) and C-Kit-D816V (SEQ ID NO: 10), but for other peptides (for example, NRAS-Q61R (SEQ ID NO: 7), KRAS-G12D (SEQ ID NO: 1), KRAS-G12R (SEQ ID NO: 4), KRAS-G13D (SEQ ID NO: 5), etc.), shorter length peptides each of which has an ability to activate the neoantigen-specific T cells can also be identified in the same manner.

TABLE 2

| | Mutation | | |
|---|---|---|---|
| Gene | name | Peptide sequence * | SEQ ID NO. |
| PIK3CA | PIK3CA- | EALEYFMKQMNDA [symbol] | SEQ ID NO: 8 |
| | H1047R | HGGWTTKMDWIFH | |
| | | EALEYFMKQMNDA [symbol] H | SEQ ID NO: 11 |
| | | ALEYFMKQMNDA [symbol] HG | SEQ ID NO: 12 |
| | | LEYFMKQMNDA [symbol] HGG | SEQ ID NO: 13 |
| | | EYFMKQMNDA [symbol] HGGW | SEQ ID NO: 14 |
| | | YFMKQMNDA [symbol] HGGW | SEQ ID NO: 15 |
| | | FMKQMNDA [symbol] HGGWT | SEQ ID NO: 16 |
| | | MKQMNDA [symbol] HGGWTT | SEQ ID NO: 17 |
| | | KQMNDA [symbol] HGGWTTK | SEQ ID NO: 18 |
| | | QMNDA [symbol] HGGWTTK | SEQ ID NO: 19 |
| | | MNDA [symbol] HGGWTTKM | SEQ ID NO: 20 |
| | | NDA [symbol] HGGWTTKMD | SEQ ID NO: 21 |
| | | DA [symbol] HGGWTTKMDW | SEQ ID NO: 22 |
| | | A [symbol] HGGWTTKMDWI | SEQ ID NO: 23 |
| C-Kit | C-Kit-D816V | G [symbol] ITKICDFGLAR [symbol] | SEQ ID NO: 10 |
| | | IKNDSNYVVKGNA | |
| | | FGLAR [symbol] IKNDSNYVV | SEQ ID NO: 24 |
| | | GLAR [symbol] IKNDSNYVVK | SEQ ID NO: 25 |
| | | LAR [symbol] IKNDSNYVVKG | SEQ ID NO: 26 |
| | | AR [symbol] IKNDSNYVVKGN | SEQ ID NO: 27 |
| | | R [symbol] IKNDSNYVVKGNA | SEQ ID NO: 28 |

* The amino acid occurring mutation is underlined in the peptide.

The amino acids constituting the peptide of the present invention may be natural amino acids or amino acid analogues. Examples of the amino acid analogues include a N-acylated product, an O-acylated product, an esterified product, an acid amidated product, an alkylated product of amino acids, etc. The peptide of the present invention may be modified with the constituent amino acids or carboxyl groups thereof, etc., provided that the functions thereof are not significantly impaired. The modification includes modification by binding of the N-terminus of the peptide or the free amino group of the amino acids with a formyl group, an acetyl group, a t-butoxycarbonyl group, etc., and modification by binding of the C-terminus of the peptide or the free carboxyl group of the amino acids with a methyl group, an ethyl group, a t-butyl group, a benzyl group, etc.

The peptide of the present invention can be produced by general methods of peptide synthesis. Such methods include, for example, the method described in Peptide Synthesis, Interscience, New York, 1966; The Proteins, Vol 2, Academic Press Inc., New York, 1976; Peptide Synthesis, Maruzen Co., Ltd., 1975; Basics and Experiments of Peptide Synthesis, Maruzen Co., Ltd., 1985; Development of Pharmaceuticals, Continued Vol. 14 and Peptide Synthesis, Hirokawa-books, Co., Ltd., 1991 (the literatures are incorporated in the present specification by reference), etc.

Use of Peptide

When the peptide thus obtained in the present specification is administered to a living body, such as a cancer patient, it can activate and proliferate T-cells, more specifically, at least CD4-positive helper T cells, which recognize and specifically react to a complex of a neoantigen-derived peptide or the neoantigen from which the peptide originated with an HLA Class II molecule on a cancer cell, in the living body, and it can be used as a peptide vaccine against cancer.

When the neoantigen-derived peptide of the present invention is used as a peptide vaccine, this peptide can be administered to mammals including a human, but it can also be administered to non-human animals, which include, but are not limited to, for example, a pig, a cow, a horse, a dog, a cat, a mouse, a rat, a rabbit, and a guinea pig. More specifically, it is preferably administered to a human.

In the present invention, the neoantigen-derived peptide can be used as a peptide vaccine against cancer for prophylactic treatment and therapeutic treatment. An object of treatment of such cancers includes, for example, tumor lesion shrinkage or reduction of growth, reduction of new lesion appearance, prolonged survival, improvement of tumor-related subjective and objective symptoms or reduction of exacerbation, reduction of metastasis, prevention of recurrence, etc.

The peptide vaccine containing the neoantigen-derived peptide of the present invention can be administered to a patient by, for example, intradermal or subcutaneous administration. The peptide vaccine containing the neoantigen-derived peptide of the present invention may contain a pharmaceutically acceptable salt, carrier, etc., so as to be suitable for such administration. The salt includes, but not limited to, alkali metal bicarbonates, such as sodium chloride and sodium hydrogen carbonate. The agent of the present invention is preferably administered by dissolving it in water, etc., so as to be isotonic with plasma. The carrier includes cellulose, polymerized amino acids, albumin, etc., and if necessary, a carrier to which the peptide used in the present invention is bound can also be used.

The peptide vaccine containing the neoantigen-derived peptide of the present invention may be formulated in a formulation such as a liposome formulation, a particulate formulation bound to beads having diameters of several um, a formulation bound to a lipid, etc. Moreover, when the neoantigen-derived peptide of the present invention is used as a peptide vaccine, it can also be administered with immunopotentiators known to be conventionally used for vaccine administration such as an incomplete Freund's adjuvant (for example, ISA-51 et al., SEPPIC Inc.), polysaccharides, such as pullulan, a complete Freund's adjuvant, BCG, Alum, GM-CSF, IL-2, CpG so as to effectively activate the immune response.

The dose of the peptide vaccine containing the neoantigen-derived peptide of the present invention can be appropriately adjusted according to the state of the disease, the age, and the body weight of each patient, etc., and the amount of the peptide in a single dose drug is usually 0.0001 mg to 1,000 mg, preferably 0.001 mg to 100 mg, more preferably 0.01 mg to 10 mg, and still more preferably 0.1 to 5 mg or 0.5 to 3 mg. It is preferably administered repeatedly once every few days, once every few weeks, or once every few months and so on.

Moreover, the peptide of the present invention can also activate and proliferate immune cells including T cells that specifically react with the peptide of the present invention or the neoantigen from which the peptide originated, by contacting the peptide of the present invention with lymphocytes collected from the living body under ex vivo culture conditions. This peptide can activate and proliferate at least CD4-positive T cells, and can further activate any or a plurality of immune cells, such as CD8-positive T cells, y6T cells, NK cells, NKT cells, dendritic cells, macrophages, etc., in addition to CD4-positive T cells. The aforementioned immune cells activated ex vivo can also be used for cancer immunotherapy, such as adoptive immunotherapy that damages cancer cells, by administering to cancer patients.

The peptide of the present invention can be brought into contact with mammalian cells including a human as in the case of administration in vivo, and it can also be brought into contact with animals other than a human.

The peptide of the present invention can also be used to prepare antigen-presenting cells for activating and proliferating cancer-reactive CD4-positive T cells or CD8-positive CTLs in cancer patients. Accordingly, it is possible to provide a method for preparing an antigen-presenting cell by contacting a cell having an antigen-presenting ability with the peptide of the present invention. Antigen-presenting cells can be prepared, for example, by culturing cells having an antigen-presenting ability derived from a cancer patient and the peptide of the present invention to contact them with each other and binding and presenting the peptide to the HLA molecule of the cell, or by introducing a vector capable of expressing the peptide into cells having an antigen-presenting ability, derived from a cancer patient to express it. The antigen-presenting cells prepared in this way can used for cancer immunotherapy by administering in vivo for activating and proliferating cancer-responsive CD4-positive T cells or CD8-positive CTLs in vivo.

The cell having an antigen-presenting ability is, for example, a dendritic cell. Dendritic cells derived from a patient can be acquired, for example, by separating culture plate adherent cells from the PBMCs collected from a patient and culturing the cells in the presence of an IL-4 and GM-CSF for about 1 week. The antigen-presenting cells prepared by the above method can activate and proliferate CD4-positive T cells or CD8-positive CTLs that specifically recognize the complex of the peptide and the HLA molecule presented on the surface of cancer cells, and can promote the activation and proliferation of cancer-reactive CD4-positive T cells or CD8-positive CTLs in a cancer patient's body when administered to the patient. Therefore, the antigen-presenting cells prepared by use of the peptide of the present invention can be used as a medicament for treating cancer.

Identification of T Cell Receptor and Utilization Thereof

Moreover, in the present invention, the full length or the part of the amino acid sequence of the T cell receptor (TCR) having reactivity with the peptide or the gene encoding the amino acid sequence thereof, can also be identified from the neoantigen-specific T cell clone thus obtained. The TCR is a dimer composed of an a chain and a r3 chain, or a y chain and a 6 chain, and in one embodiment of the present invention, the full length or a part of the amino acid sequence of the TCRa chain or TCRI3 chain or the gene encoding the amino acid sequence thereof can be isolated, and from the structure of each TCR chain, the amino acid sequences of the complementarity determining region (CDR) of the variable region (V region), i.e., CDR1, CDR2, and CDR3, or the gene encoding the full length or a part of the amino acid sequences thereof can be isolated, and more specifically the amino acid sequences of the complementarity determining regions (CDR) of the TCRa chain or the TCR(3 chain, CDR1a, CDR2a, CDR3a, CDR113, CDR2I3, CDR3 f3 or the gene encoding the amino acid sequences thereof can be isolated.

T cells in which the TCR gene can be modified (TCR-T) can be produced by introducing the TCR gene thus obtained into T cells. For example, plasmid vector(s) or virus vector(s) (a retrovirus vector or a lentivirus vector) prepared by inserting the TCRa chain gene (full length or a part) and the TCRI3 chain gene (full length or a part), identified from the neoantigen-specific T cells in the present invention, can be introduced into T cells derived from a cancer patient or a healthy person to prepare a T cell line in which the TCR gene is modified. The prepared TCR gene-modified cell line can exhibit specificity for the neoantigen, which is reactive to the antigen-presenting cell presenting the neoantigen of the present invention and the peptide derived from the neoantigen of the present invention.

The present invention will be specifically exemplified with reference to the following Examples. The Examples exemplified below do not limit the present invention by any method.

### EXAMPLES

### Example 1: Design and Synthesis of Peptides Targeting Neoantigens Derived from Driver Mutation

In this example, according to the database search and literature search, 27-mer peptides containing a mutated amino acid site of known driver mutations (6 mutations in KRAS, NRAS, PIK3CA, and C-Kit genes) (peptides containing 11 to 13 amino acids on the N-terminal side and 13 to 15 amino acids on the C-terminal side, centering a mutated amino acid site therein) were selected, and based thereon, total of 15 peptides such as 10 neoantigen peptides (SEQ ID NO: 1 to SEQ ID NO: 10) and 5 wild-type genes were designed and synthesized (FIG. 1). FIG. 1 shows the Mutation IDs of the genetic mutations in the ICGC (International Cancer Genome Consortium) database and the sequences of the peptides that were synthesized so as to contain respective mutations. The 15 peptides shown in FIG. 1 were synthesized by Sigma-Aldrich Japan LLC.

The synthesized peptide powder was weighed with an electronic balance, and 10 mg/mL of dimethyl sulfoxide (DMSO, Sigma-Aldrich Japan LLC., D8418) was added thereto. The peptide was dissolved by stirring with a vortex mixer, dispensed and stored in a low temperature chamber set at −20° C.

### Example 2: Immunogenicity Evaluation of Each Peptide

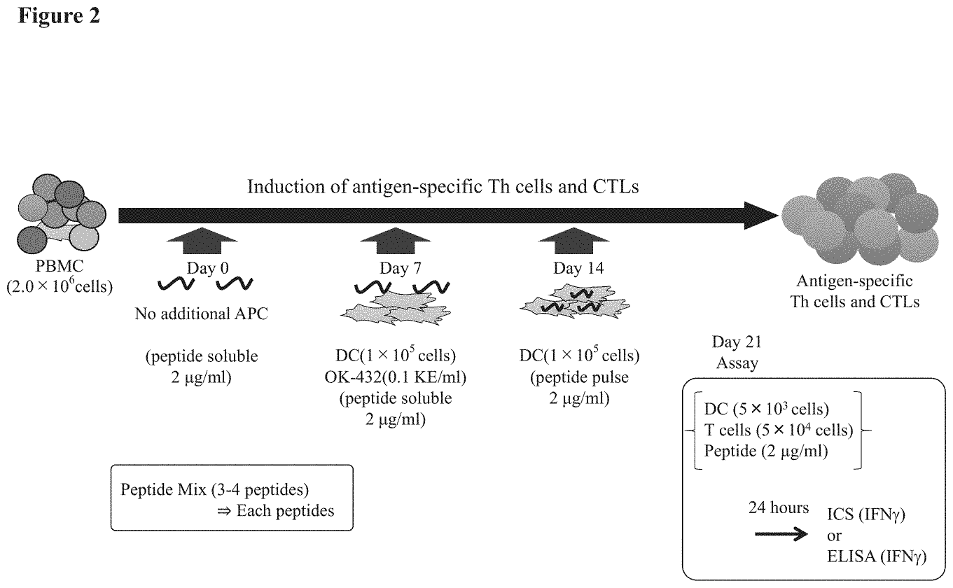

In this example, the immunogenicity of each peptide synthesized in Example 1 was evaluated by the steps shown in FIG. 2.

(2-1) Activation of Antigen-Specific T Cells and Culture of Dendritic Cells (DC)

Peripheral blood mononuclear cells (PBMCs) that were those of a lot containing HLA-A*24:02 or A*02:01 of purified normal human PBMCs (Precision Bioservice, 93000-10M or -50M), were selected and used. Alternatively, PBMCs were separated and collected by density gradient centrifugation from peripheral blood provided by 10 healthy volunteers (including those of HLA-A*24:02 or A*02:01) recruited at the Kanagawa Cancer Center Research Institute, and were used in the experiment.

Healthy human PBMCs of 2×10⁶ cells were cultured (a CO2 incubator; 5% CO2 and at 37° C.) for 7 days in the presence of the peptides to be evaluated (2, 2.5 or 5 μg/mL, and in the case of Mix (described later), 2 lag/mL for each peptide), and then the cells were collected. AIM-V medium (Thermo Fisher Scientific K.K., 12055-091) supplemented with a 5% human serum (NIP Biomedicals, 2931949) was used as medium.

On the other hand, the PBMCs of the same lot were cultured in the presence of a GM-CSF and IL-4 for 7 days to differentiate into dendritic cells (DC), and a part thereof was cryopreserved.

Cell stimulation for evaluation of immunogenicity was carried out according to the method outlined in FIG. 2. Namely, the PBMCs stimulated with the peptide for 7 days were collected and co-cultured in the presence of DC (1×10⁵ cells) treated with a mitomycin C (60 μg/mL, Kyowa Kirin Co., Ltd.), each peptide (2, 2.5 or 5 μg/mL), and 0.1 KE/mL of OK-432 (for injection of Picibanil, Chugai Pharmaceutical Co., Ltd.). An IL-2 (PeproTech, Inc., AF-200-02) was added at 10 IU/mL on the second day of the co-culture (Day 9 with the stimulation start date as Day 0), and the cells were further cultured for 5 days.

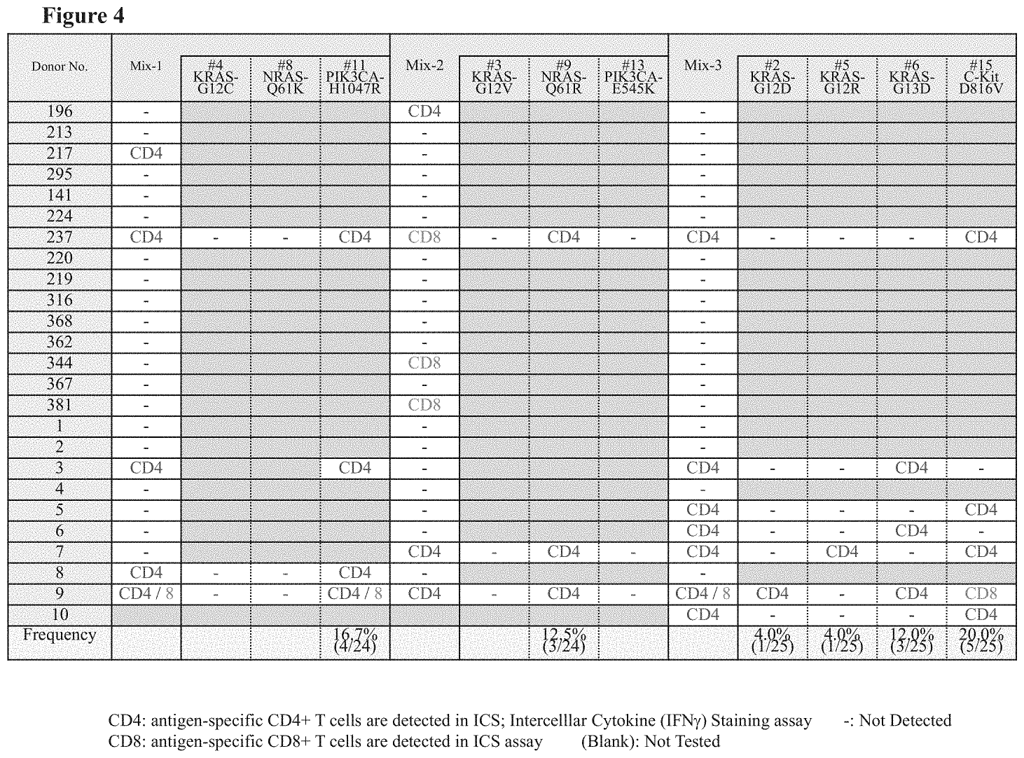

After culturing (Day 14), the cells were collected, the cryopreserved DC was thawed, and the cells were co-cultured again in the presence of the same concentration of each peptide for 7 days. The cells cultured for a total of 21 days were collected, and activation of antigen-specific T cells was confirmed by intracellular cytokine staining (ICS) or IFNy ELISA. For evaluation, the PBMCs for 24 donors (of Mix-1 and Mix-2) or the PBMCs for 25 donors (of Mix-3) (the donor numbers 1 to 10 are derived from healthy volunteers, and the donor numbers with 3 digits were purchased from Precision Bioservice, Inc.), were used. In this study, 10 kinds of peptides designed from the sequences containing the driver mutations in Example 1 and synthesized (SEQ ID NO: 1 to SEQ ID NO: 10) were mixed to the following mixtures containing 3 to 4 kinds of peptides (see FIG. 4): Mix-1: a mixture of KRA S-G 1 2C (SEQ ID NO: 3), NRAS-Q61K (SEQ ID NO: 6) and PIK3CA-H1047R (SEQ ID NO: 8); Mix-2: a mixture of KRAS-G12V (SEQ ID NO: 2), NRAS-Q61R (SEQ ID NO: 7), and PIK3CA-E545K (SEQ ID NO: 9); and Mix-3: a mixture of KRAS-G12D (SEQ ID NO: 1), KRAS-G12R (SEQ ID NO: 4), KRAS-G13D (SEQ ID NO: 5), and C-Kit-D816V (SEQ ID NO: 10). First, activation of antigen-specific T cells by stimulation with each Mix was observed, and when the activation was observed, the T cells were stimulated in the presence of each peptide alone to identify a peptide having immunogenicity. The presence or absence of activation was determined by the ICS (IFNy) or ELISA (IFNy in the culture supernatant).

(2-2) Intracellular Cytokine Staining (ICS)

Intracellular cytokine staining (ICS) was carried out according to the following procedure. The cells (5.0×10⁴ cells) cultured for 21 days in the presence of the peptide and collected in the present Example, and antigen-presenting cells (APC) (autologous DC; 5×10³ cells) were placed in a 96-well U bottom plate and cultured for 2 hours in the presence of the peptide. 10 μg/mL of Brefeldin A (Merck KGaA, B7651) was added to the cells to culture for 20 to 24 hours. The cells after the culture were collected, to which an APC-labeled anti-CD3 antibody (Biolegend, 300412), a FITC-labeled anti-CD4 antibody (BD Pharmingen, 555346), and an APC-Cy7-labeled anti-CDS antibody (TONBO, 25-0088-T100) were added to stain the cells for 15 minutes under the condition of 4° C. The stained cells were treated with a BD Cytofix/Cytoperm (BD Pharmingen, 51-2090KZ), to which the PE-Cy7-labeled anti-IFNy antibody (BD Pharmingen, 557643) was added to the cells to stain for 40 minutes under the condition of 4° C. After washing the stained cells, the expression of each cell surface antigen and cytokine was analyzed with the BD FACSCanto™ II.

(2-3) IFNy ELISA

Further, IFNy ELISA was carried out according to the following procedure. The cells (5.0×10⁴ cells) collected after culturing for 21 days in the presence of the peptide and collected in the present Example, and APC (autologous DC; 5×10³ cells) were placed in a 96-well U bottom plate and cultivated in the presence of an antigen for 20 to 24 hours. The supernatant after culturing was collected, in which the amount of IFNy in the supernatant was quantified by the ELISA method. Assay Diluent (BD Pharmingen, 51-2641KC) was added to an ELISA plate (Corning, 9018) on which the anti-IFNy capture antibody (BD, 51-26131E) was immobilized, which was incubated at room temperature for 1 hour. After discarding and washing the Assay Diluent, a sample to be measured (culture supernatant) diluted to an appropriate magnification was added, which was incubated at room temperature for 90 minutes. After discarding and washing the sample, an anti-IFNy detection antibody (Detection Antibody Biotin Anti-Human IFNy) (BD Pharmingen, 51-26132E) and Streptavi din-horseradish peroxidase conjugate (Say-HRP) (BD Pharmingen, 51-9002208) were added to the cells, which was incubated for 45 minutes at room temperature. After discarding and washing the antibody solution, TMB substrate liquid (prepared by using the Substrates A and B (BD Pharmingen, 51-2606KZ and 51-2607KZ)) was added. After confirming the color development, the reaction was stopped with addition of Stop Solution (BD Pharmingen, 51-2608KZ), and an absorbance (0D450) was measured with a plate reader.

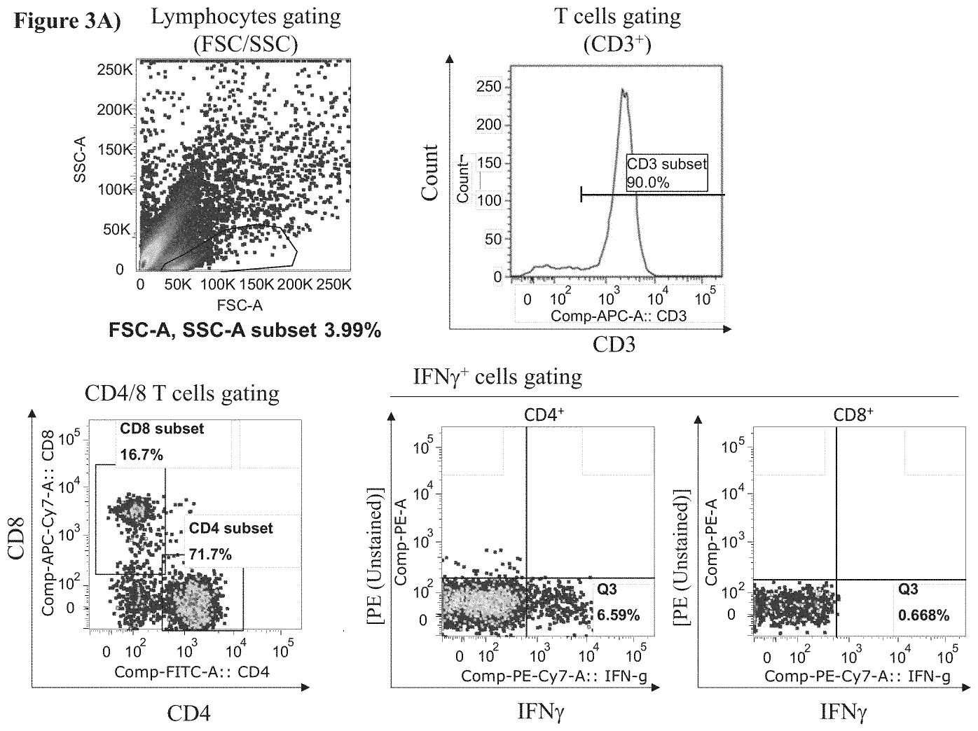

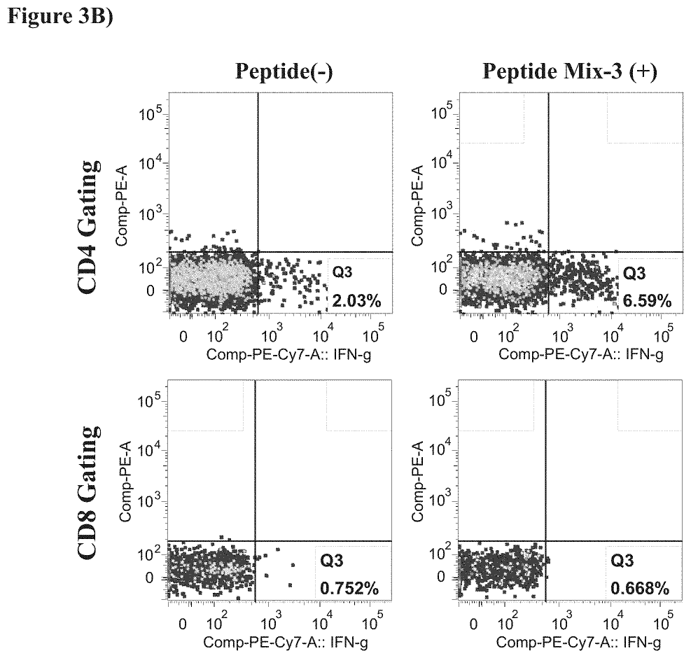

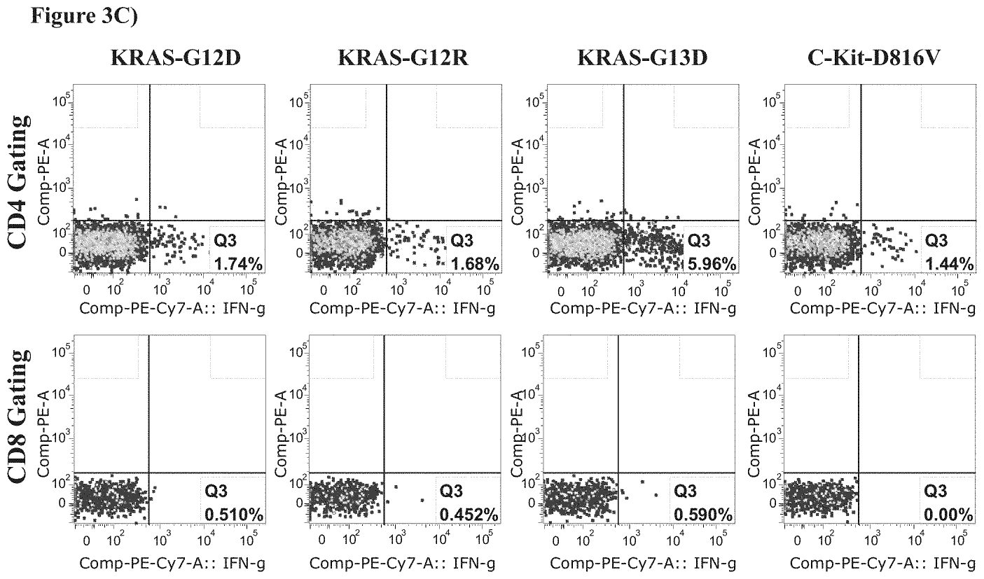

(2-4) Evaluation of immunogenicity of neoantigen candidate peptides FIG. 3 shows the results of observation of antigen-specific T cell activation by the ICS. FIG. 3 (A) shows the gating steps. First, a population of cells with lymphocyte-like morphology was gated with deployment of FSC (forward scattered light)/SSC (side scattered light), and then the CD3 (T cell marker)-positive T cell population was gated. The CD4 or CD8-positive T cell population was further gated from the CD3-positive population, and the ratio of the CD4 or CD8-positive IFNy production-positive population in each population was calculated. FIG. 3 (B) shows the case where the PBMCs derived from the donor No. 6 selected from 25 healthy subjects (FIG. 4) was stimulated and cultured with Mix-3 (a mixture of KRAS-G12D (SEQ ID NO: 1), KRAS-G12R (SEQ ID NO: 4), KRAS-G13D (SEQ ID NO: 5), C-Kit-D816V (SEQ ID NO: 10), FIG. 4), is described. CD4-positive IFNy-producing cells were detected by Mix-3 stimulation. FIG. 3 (C) shows the results of stimulating the cells with each of four peptides that constitutes Mix-3. The cells were found to respond only to KRAS-G13D (SEQ ID NO: 5). In such a way, antigens having immunogenicity were identified relating to T cells derived from each donor. The above description demonstrates the results of stimulation and culture with Mix-3, but even in the cases of Mix-1 and Mix-2, CD4-positive IFNy-producing cells were detected by the similar method, and a reactive neoantigen-derived peptide antigen could be identified.

The ratio of the IFNy-positive cell population was calculated in the CD4 or CD8-positive cells. Analysis was conducted under both conditions of presence of antigen and absence of antigen, and the reaction was considered to be positive in the case where the IFNy-positive cell population (%) [i.e., ratio of IFNy-positive cells in CD4 or CD8-positive cells] under the condition of presence of the antigen is 1.3% or larger and the IFNy-positive cell population (%) [i.e., ratio of IFNy-positive cells in CD4 or CD8-positive cells] under the condition of absence of the antigen is 1.0% or larger.

As a result of stimulating with Mix-1 (i.e., a mixture of KRAS-G12C (SEQ ID NO: 3), NRAS-Q61K (SEQ ID NO: 6) and PIK3CA-H1047R (SEQ ID NO: 8)) (FIG. 4), activation of antigen-specific T cells (CD4 positive) was observed in the samples of 5 donors among 24 donors. The T cells derived from 1 donor lost their proliferative capacity during the culture step and could not proceed to detailed analysis (donor No. 217). As a result of re-analyzing the T cells derived from the remaining 4 donors by using each peptide, it was confirmed that all of them specifically responded to PIK3CA-H1047R. PIK3CA-H1047R (SEQ ID NO: 8) had immunogenicity (i.e., the effect of activating peptide-specific T cells) in 4/24 samples (16.7%). All antigen-specific T cells activated in the 4 donors were CD4 positive, and activation of CD8-positive cells was also observed in the donor No. 9. The PIK3CA-H1047R (SEQ ID NO: 8) was found to exhibit immunogenicity as both the HLA Class I epitope and the Class II epitope.

As a result of stimulating with the Mix 2 (i.e., a mixture of KRAS-G12V (SEQ ID NO: 2), KRAS-Q61R (SEQ ID NO: 7) and PIK3CA-E545K (SEQ ID NO: 9)), activation of antigen-specific T cells was observed in 6 of 24 donor samples (FIG. 4). The T cells derived from 3 of 6 donor samples were proliferated and analyzed using a single peptide, resulting in the specific reaction with NRAS-Q61R (SEQ ID NO: 7) in all of them. Moreover, they were all CD4-positive T cells. The NRAS-Q61R (SEQ ID NO: 7) was found to exhibit immunogenicity in 3/24 samples (12.5%).

As a result of stimulating with the Mix 3 (i.e., a mixture of KRAS-G12D (SEQ ID NO: 1), KRAS-G12R (SEQ ID NO: 4), KRAS-G13D (SEQ ID NO: 5) and C-Kit-D816V (SEQ ID NO: 10)), activation of antigen-specific T cells was observed in 7 of 25 donor samples (FIG. 4). As a result of analysis using a single peptide, in detail, the antigen-specific T cells for KRAS-G12D and KRAS-G12R was observed in 1 donor (1/25, 4.0%), those for KRAS-G13D was observed in 3 donors (3/25, 12.0%), and those for C-Kit-D816V was observed in 5 donors (5/25, 20.0%). Of the C-kit-D816V-specific T cells activated in 5 donors, those derived from 4 donors were CD4 positive, whereas activation of CD8-positive cells was observed in 1 donor (No. 9). The C-Kit-D816V was found to exhibit immunogenicity as both the HLA Class I epitope and the Class II epitope.

From these results, activation of CD4-positive T cells was observed with the peptides of KRAS-G12D (SEQ ID NO: 1), KRAS-G12R (SEQ ID NO: 4), KRAS-G13D (SEQ ID NO: 5), NRAS-Q61R (SEQ ID NO: 7), PIK3CA-H1047R (SEQ ID NO: 8) and C-Kit-D816V (SEQ ID NO: 10), and among these, in particular 4 peptides derived from PIK3CA-H1047R (SEQ ID NO: 8), NRAS-Q61R (SEQ ID NO: 7), KRAS-G13D (SEQ ID NO: 5) and C-Kit-D816V (SEQ ID NO: 10) were found to exhibit immunogenicity in the samples derived from healthy people with a high frequency exceeding 10% (FIG. 4).

### Example 3: HLA Class II Restriction of Neoantigen Candidates in which Immunogenicity was Confirmed

In this example, HLA Class II restriction of the neoantigen candidates in which immunogenicity was confirmed in Example 2 was revealed.

(3-1) HLA-Class II Blocking Assay

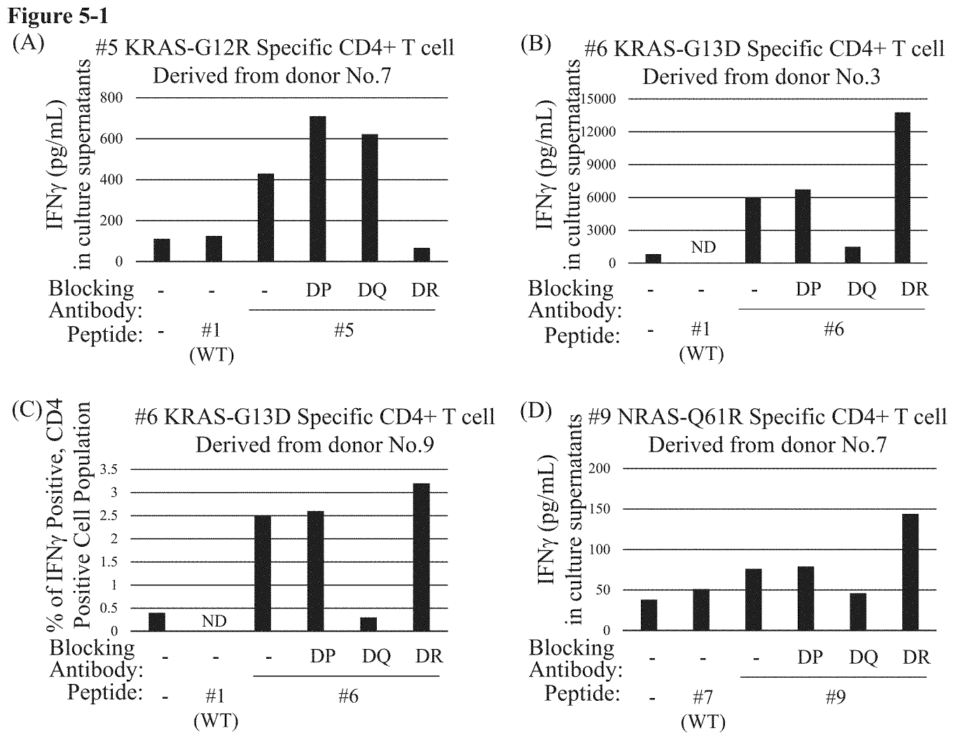

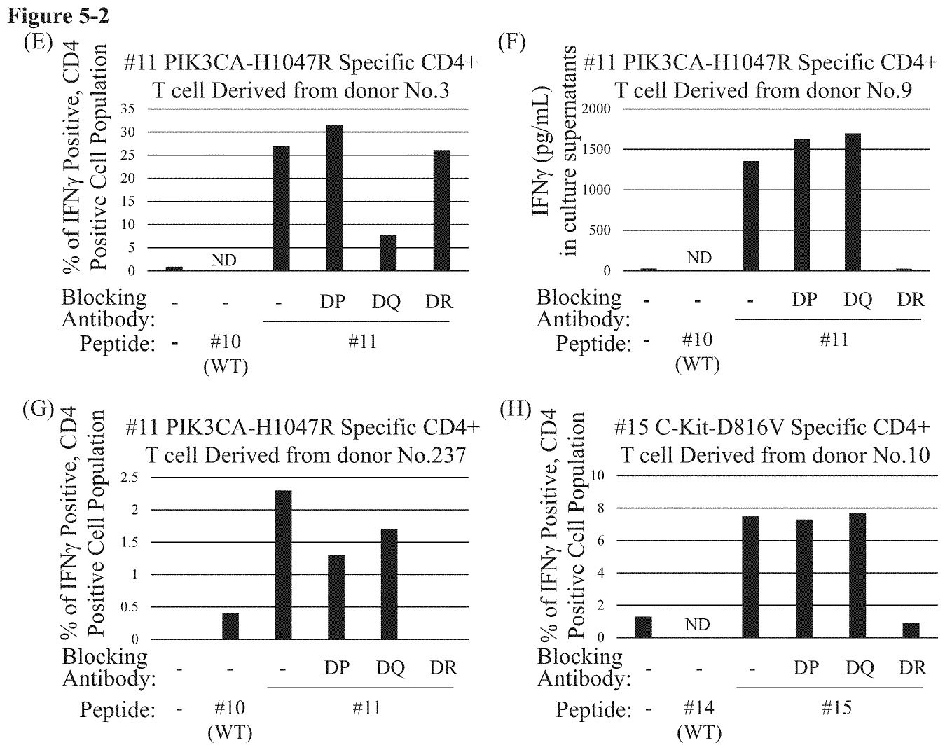

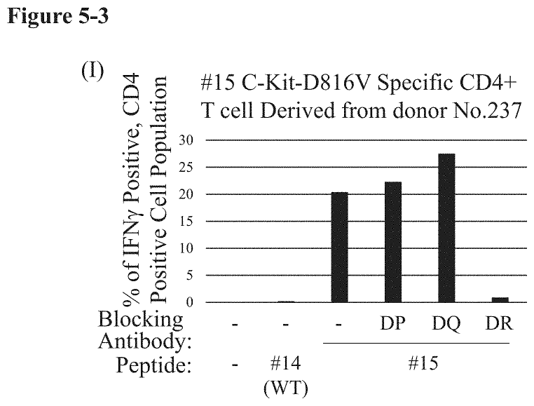

The HLA Class II restriction for the CD4-positive T cells that could be continuously cultured (those derived from donors No. 3, No. 7, No. 9, No. 10, and No. 237 individuals) among those with antigen specificity confirmed by the ICS or IFNy ELISA Assay in Example 2 was confirmed by the Blocking Assay using the anti-HLA Class II antibody. Specifically, the residual CD4-positive T cells with antigen specificity confirmed was cultured in AIM-V medium supplemented with a 5% human serum and 20 U/mL of IL-2 for 1 day or longer, and then the ICS or IFNy ELISA was conducted by employing the same method as in (2-2) or (2-3) under the conditions of addition of each anti-HLA Class II antibody [i.e., 200 μg/mL of anti-HLA-DP (BRAFB6, Santa Cruz Biotechnology, SC-33719), 1 mg/mL of HLA-DQ (SPV-L3, Abcam, ab23632), or 1 mg/mL of HLA-DR (G46-6, BD Pharmingen, 555809)], together with peptides. The obtained results are shown in FIG. 5 (FIG. 5-1 to FIG. 5-3). The ND in the figure denotes “No Data”.

The donor No. 7-derived KRAS-G12R-specific T cells did not respond to the wild-type (KRAS-WT) of the antigen (i.e., KRA S-G12R) used for activation, but responded to the KRAS-G12R (SEQ ID NO: 4). This reaction was not inhibited by the anti-DP antibody or anti-DQ antibody, but was inhibited by the addition of the anti-DR antibody (FIG. 5-1 (A)), from which the reaction of T cells to the antigen was found to be DR restricted. Similarly, it was found that the donor No. 3 and No. 9-derived KRAS-G13D-specific T cells was DQ-restricted (FIGS. 5-1 (B) and (C)), and the donor No. 7-derived NRAS-Q61R-specific T cells was DQ-restricted (FIG. 5-1 (D)), the donor No. 3-derived PIK3CA-H1047R-specific T cells was DQ-restricted (FIG. 52 (E)), the donor No. 9 and 237-derived PIK3CA-H1047R-specific Target T cells was DR-restricted (FIGS. 5-2 (F) and (G)), and the donor No. 10 and 237-derived C-Kit-D816V-specific T cells was DR-restricted (FIG. 5-2 (H) and FIG. 5-3 (I)).

(3-2) Analysis of HLA Restriction of Antigen-Specific T Cells by Using LCL (Lymphoblastoid Cell Line)

Subsequently, for T cells with DP/DQ/DR restriction which could be identified by the Blocking Assay in (3-1) (those derived from the donors No. 3, No. 7, No. 9, and No. 10 individuals), an attempt was made to identify an allele of DP/DQ/DR using an allogeneic B cell line as APC for specificity confirmation. The LCL (Lymphoblastoid Cell Line) was created by infecting EB virus (B95-E cells; the culture supernatant of JCRB cell bank JCRB9123) to non-adherent cells collected when the DC was prepared from each healthy person's PBMCs.

For T cells with antigen specificity confirmed by the ICS, antigen specificity was confirmed by the ICS or ELISA using the allogeneic LCL as APC under the condition that the ratio of APC:T cells was 2 (2 to 10×10⁴ cells/well): 1 (1 to 5×10⁴ cells/well).

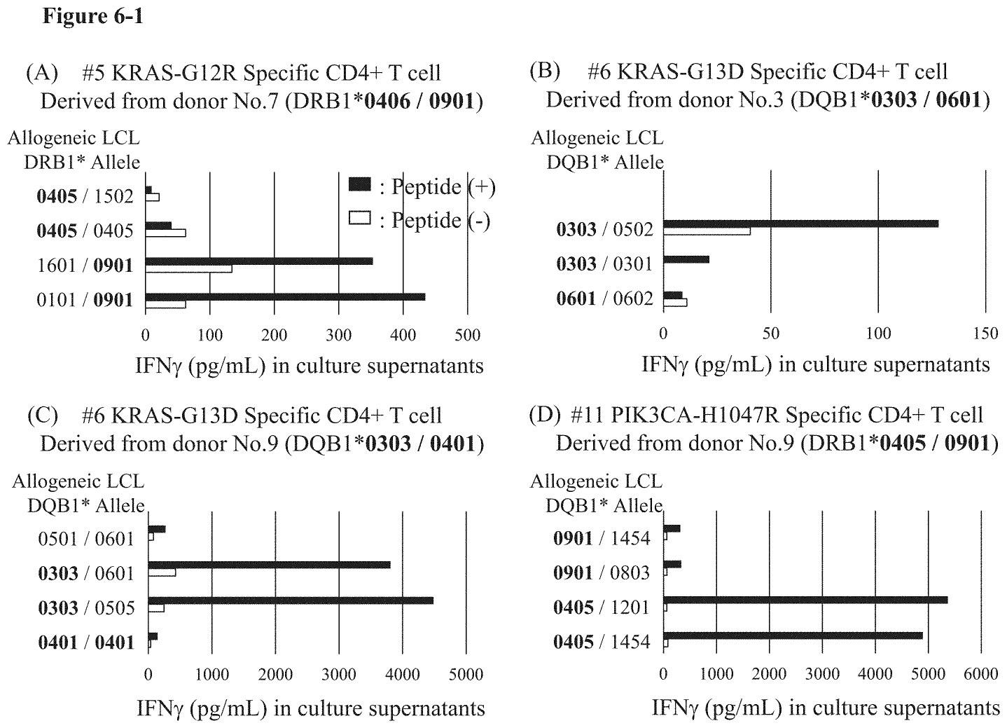

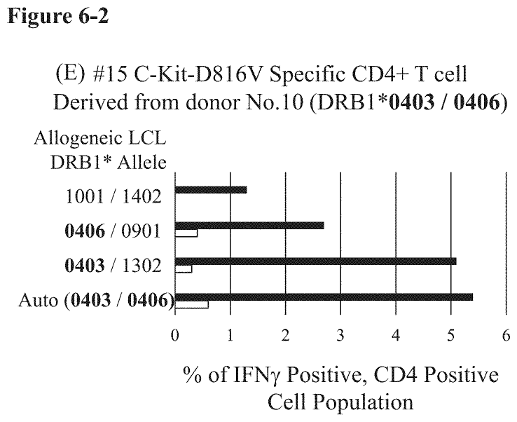

The donor NO. 7-derived KRAS-G12R-specific T cells exhibited a reaction to the antigen in the presence of the APC having DRB1*0901, indicating that they were the allele restricted (FIG. 6-1 (A)). Similarly, it was found that the donor No. 3 and No. 9-derived KRAS-G13D-specific T cells were both DQB1*0303 restricted (FIGS. 6-1 (B) and (C)), the donor No. 9-derived PIK3CA-H1047R-specific T cells were DRB1*0405 restricted (FIG. 6-1 (D)), and the donor NO. 10-derived C-Kit-D816V-specific T cells were DRB1*0403 or 0406-restricted (FIG. 6-2 (E)).

### Example 4: Identification of Epitope Site of Neoantigen Candidates (Epitope Mapping)

In this example, the epitope sites of the neoantigen candidate peptides were identified (epitope mapping).

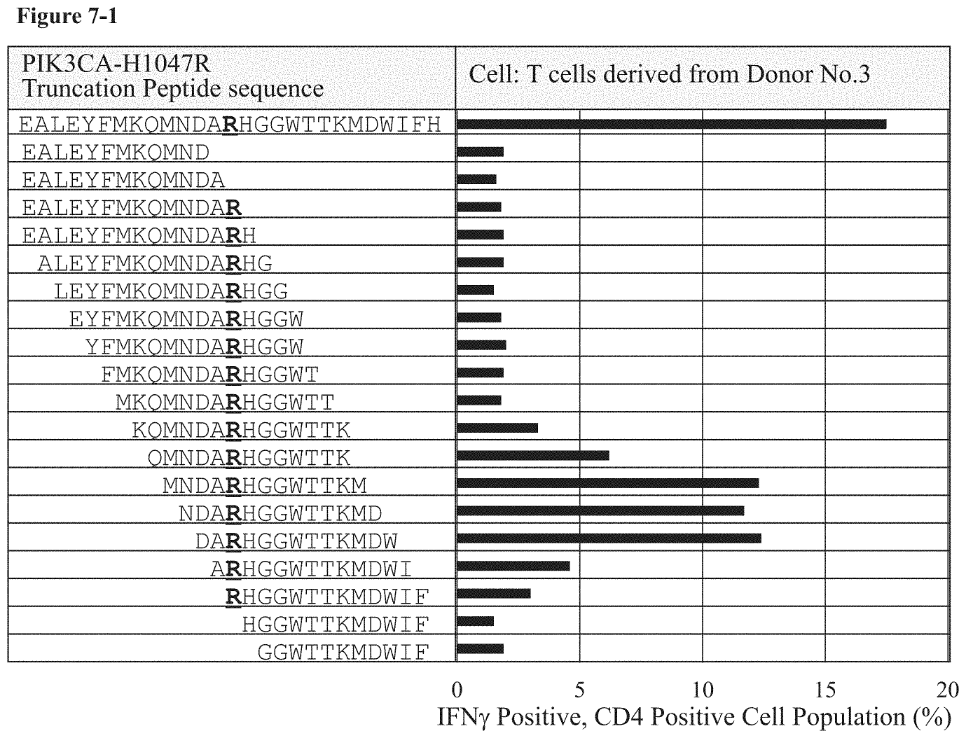

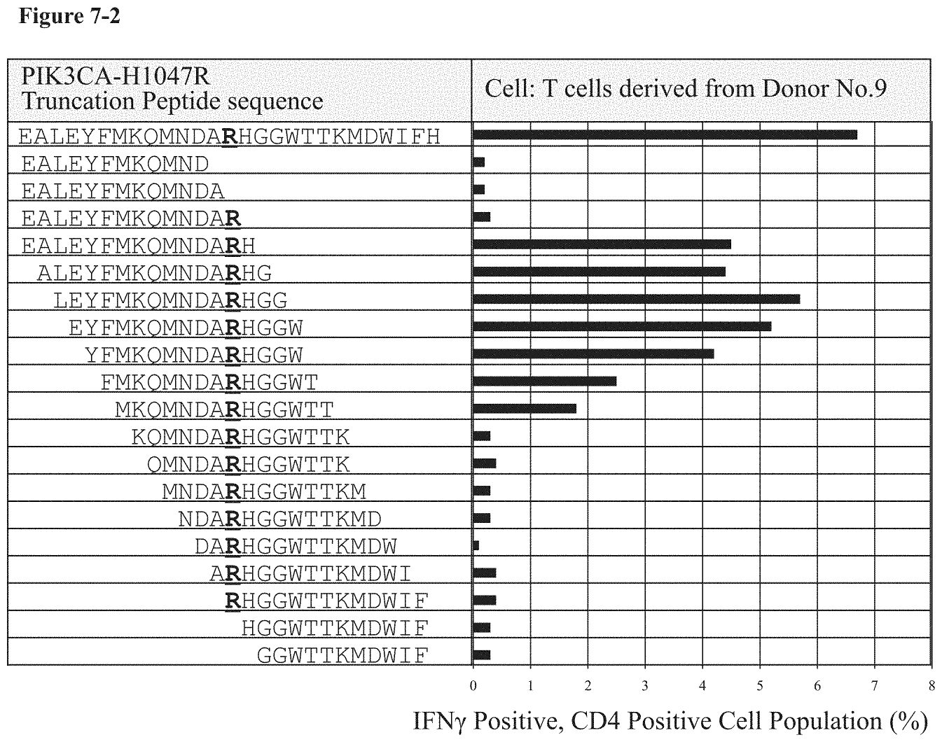

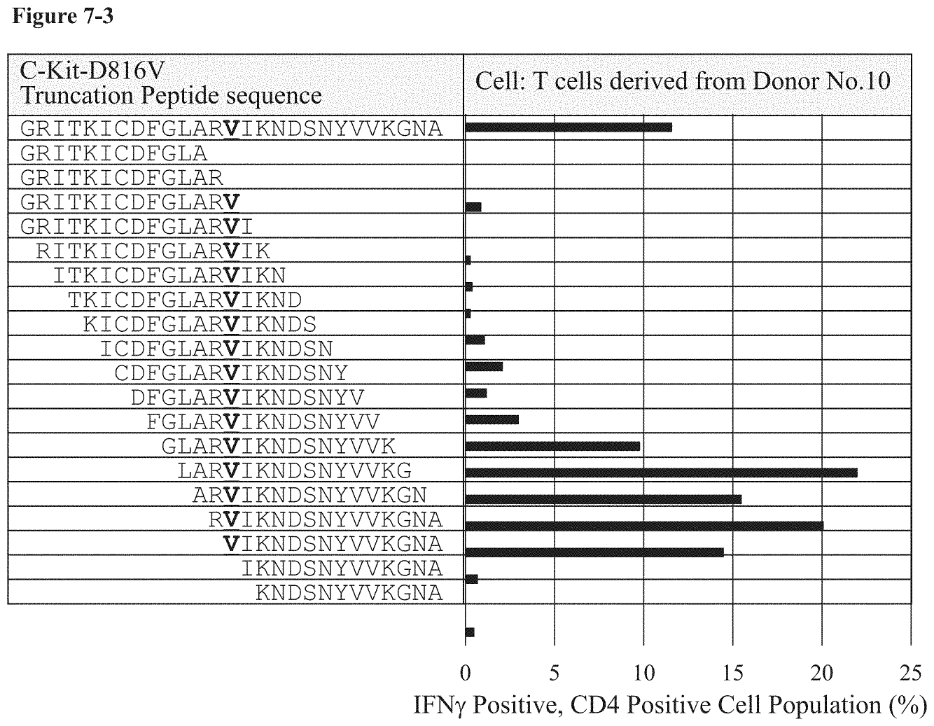

Among each peptide confirmed to activate antigen-specific T cells, 12- to 15-mer of overlapping peptide was synthesized relating to the PIK3CA-H1047R (SEQ ID NO: 8) and the C-Kit-D816V (SEQ ID NO: 10) (Sigma-Aldrich Japan LLC.). For each antigen-specific T cell, the activation ability of each overlapping peptide was confirmed by ICS using an autologous DC as APC. The results are shown in FIG. 7 (FIG. 7-1 to FIG. 7-3).

Since some of the PIK3CA-H1047R and C-Kit-D816V-specific T cells could be maintained and proliferated for a long period of time, the epitope sites were then identified by an epitope mapping method. Based on the total length of 27 amino acids of PIK3CA-H1047R (SEQ ID NO: 8) and C-Kit-D816V (SEQ ID NO: 10), overlapping peptides composed of 11 to 15 amino acids were synthesized, which were used as antigens to examine the reactivity of different antigen-specific T cells to those possible antigens. The epitope of the donor NO. 3-derived PIK3CA-H1047R-specific T cells was found to be 9 amino acids composed of ARHGGWTTK (FIG. 7-1). Similarly, it was found that the epitope of the donor NO. 9-derived PIK3CA-H1047R-specific T cells was 9 amino acids composed of MKQMNDARH (FIG. 7-2), and the epitope of the donor NO. 10-derived C-Kit-D816V-specific T cells was 10 amino acids composed of RVIKNDSNYV (FIG. 7-3).

### Example 5: TCR Gene Identification from Neoantigen-Specific T Cells and Confirmation of Antigen Specificity

In this example, the antigen-specific T cells were cloned, from which TCR gene sequences and amino acid sequences were identified.

For the T cell population that exhibited proliferation at the time of culturing for about 2 weeks after cloning by the limiting dilution method, the antigen specificity of the T cell population was confirmed by the ICS using the DC as APC. T cells whose specificity were confirmed were subjected to expansion culture using human B cell lines; EB-3 and Jiyoye as feeder cells, in the presence of 40 ng/mL of an anti-CD3 antibody (UCHT1, BD Pharmingen, 555330) and 120 IU/mL of IL-2.

The TCR gene of the antigen-specific T cells cloned by the method described above in this example was identified according to the method of Hamana et al.

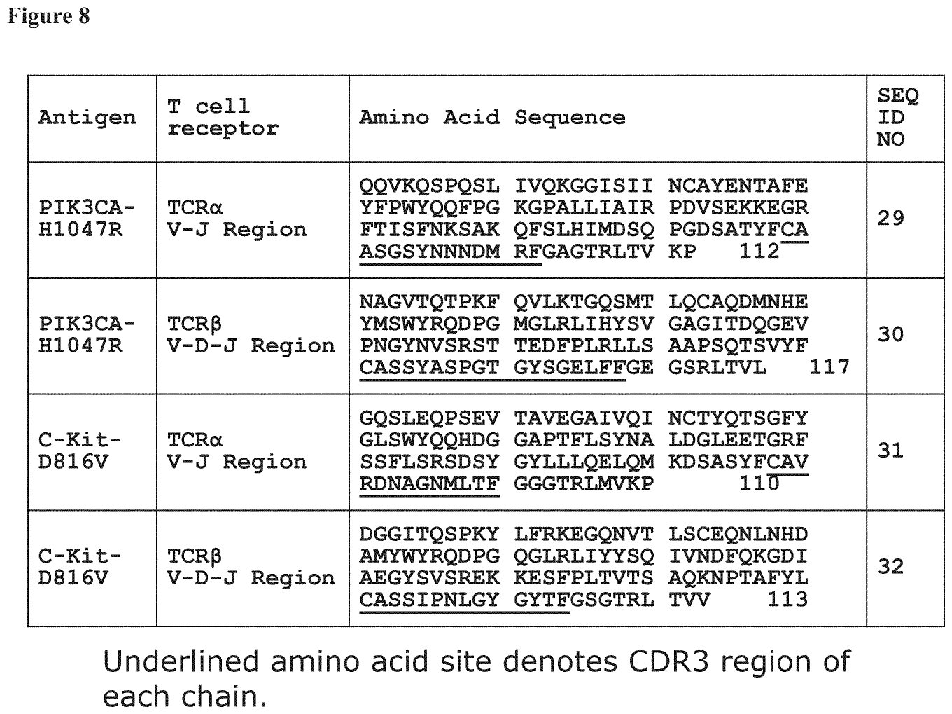

In this example, the donor NO. 9-derived PIK3CA-H1047R-specific T cells and the donor NO. 10-derived C-Kit-D816V-specific T cells were cloned by the limiting dilution method as examples, from which the TCR gene was identified by using cell clones whose the antigen reactivity were confirmed. RNA was isolated from the cells, 30 and the RT-PCR amplified products were obtained by using TCR gene-specific primers, whose nucleotide sequences were sequenced to identify the amino acid sequence of the variable (v)-joint (J) region of the TCRα chain (SEQ ID NO: 29) and the amino acid sequence of the V-diversity (D)-J region of the TCRβ chain (SEQ ID NO: 30) of the No. 9-derived PIK3CA-H1047R-specific T cells, the amino acid sequence of the V-J region of the TCRα chain (SEQ ID NO: 31) and the amino acid sequence of the V-D-J region of the TCRO chain (SEQ ID NO: 32) of the donor No. 10-derived C-Kit-D816V-specific T cells, respectively. Of these, each of these amino acid sequences was identified as follows (FIG. 8): in the donor No. 9-derived PIK3CA-H1047R-specific T cells, the amino acid sequence of the CDR3α region of the TCRα chain corresponds to the region from 89th amino acid to 102th amino acid (CAASGSYNNNDMRF) of SEQ ID NO: 29 and the amino acid sequence of the CDR3β region of the TCRβ chain corresponds to the region from 91th amino acid to 108th amino acid (CASSYASPGTGYSGELFF) of SEQ ID NO: 30; and in the donor No. 10-derived C-Kit-D816V-specific T cells, the amino acid sequence of the CDR3ot region of the TCRα chain corresponds to the region from 88th amino acid to 100th amino acid (CAVRDNAGNMLTF) of SEQ ID NO: 31, and the amino acid sequence of the CDR3β region of the TCRβ chain corresponds to the region of 91th amino acid to 104th amino acid (CASSIPNLGYGYTF) of SEQ ID NO: 32.

### Example 6: Preparation of TCR-T Cells

Via a retrovirus vector (donated by Toyama University) to which the TCRa chain gene (SEQ ID NO: 29) and TCRI3 chain gene (SEQ ID NO: 30) identified in Example 5 derived from the donor No. 9-derived PIK3CA-H1047R-specific T cells were inserted, a cell line (TCR-T cell) expressing the TCR gene was prepared by introducing the PIK3CA-H1047R-specific TCR gene into T cells derived from healthy subjects. The prepared TCR-T cells (TCR gene-expressing cell line) (5.0×10⁴ cells as T cells) and APC (donor No. 9-derived EB virus immortalized B cells; 5×10³ cells) were cultured in a 96 well U bottom plate for 20 to 24 hours in the presence of the antigen peptide (PIK3CA-H1047R (SEQ ID NO: 8)) or the wild-type peptide thereof (EALEYFMKQMNDAHHGGWTTKMDWIFH), then the cultured cells were collected, and antigen specificity was confirmed by detecting IFNy-producing cells with a flow cytometer.

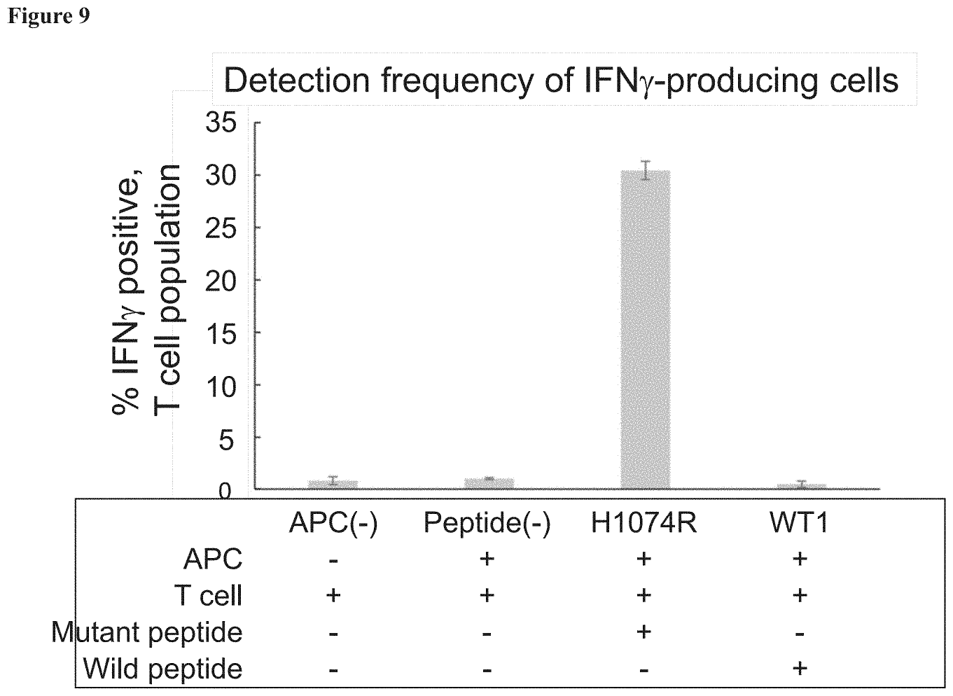

FIG. 9 shows the frequency of IFNy-producing cells detected by the flow cytometer. When PIK3CA-H1047R-specific TCR-T cells were cultured in the absence of antigen-presenting cells (APC) (denoted as “APC (−)”), when they were co-cultured with the APC (denoted as “peptide (−)”), and when they were co-cultured in the presence of wild-type peptides (denoted as “WT1”), the frequency of IFNy-producing cells was less than 1%. In contrast, the frequency of IFNy-producing cells was as high as 30.4%±0.9% when co-cultured with the APC in the presence of the antigen peptide (PIK3CA-H1047R; SEQ ID NO: 8) (denoted as “H1047R”). These results indicated that T cells into which the PIK3CA-H1047R-specific TCR genes had been introduced exhibited an immune response specific to the antigen peptide (PIK3CA-H1047R)

From this example, it was indicated that for example, when cancer cells expressing PIK3CA-H1047R (SEQ ID NO: 8) were found in an individual as an example of a neoantigen, a specific immune response could be generated for the cancer cells expressing PIK3CA-H1047R (SEQ ID NO: 8) in the individual, by introducing the TCRα chain gene (SEQ ID NO: 29) and TCRI3 chain gene (SEQ ID NO: 30) derived from the PIK3CA-H1047R-specific T cells acquired in Example 5 into T cells of the individual to prepare TCR-T cells, then proliferating them followed by returning them to the individual.

### INDUSTRIAL APPLICABILITY

The peptides with immunogenicity confirmed in the present invention can be utilized as vaccines against neoantigens derived from cancer cells for treatment or prevention of cancer. More specifically, they can be used as cancer vaccines targeting driver mutations that are highly frequently shared by cancer patients. Moreover, CD4-positive helper T cells induced by the peptides can be identified, cloned, proliferated and transferred to patients. Furthermore, by identifying the TCR gene sequences for the antigens from CD4-positive T cells and introducing the genes into the T cells, the TCR gene-modified T cells (TCR-T) can be created and used as therapeutic agents.

1. A peptide having a partial amino acid sequence comprising a mutated amino acid of a neoantigen, which is an epitope presented by an HLA Class II molecule.

2. The peptide according to claim 1, wherein the peptide activates and proliferates a CD4-positive helper T cell.

3. The peptide according to claim 1, wherein the peptide is derived from an amino acid sequence comprising a tumor-specific antigen, a cancer driver mutated protein, or a cancer passenger mutated protein.

4. The peptide according to claim 1, wherein the peptide has 9 to 27 amino acids length.

5. The peptide according to claim 1, wherein the cancer driver mutation is selected from the group consisting of PIK3CA-H1047R, C-Kit-D816V, NRAS-Q61R, KRAS-G12D, KRAS-G12R, and KRAS-G13D.

6. The peptide according to claim 1, wherein the peptide further has antigenicity as an HLA Class I-restricted epitope (having an ability to activate and proliferate a CDS-positive antigen-specific T cell).

7. The peptide according to claim 1, wherein the peptide comprises a partial sequence of an amino acid sequence selected from SEQ ID NO: 1 to 10.

8. The peptide according to claim 1, wherein the peptide consists of any one of amino acid sequences selected from SEQ ID NO: 11 to SEQ ID NO: 28.

9. A peptide vaccine against cancer which comprises the peptide according to claim 1.

10. The peptide vaccine according to claim 9, wherein the peptide vaccine activates an immune cell selected from the group consisting of a CDS-positive T cell, a CD4-positive T cell, a γδT cell, a NK cell, a NKT cell, a dendritic cell, and a macrophage.

11. A method for activating and proliferating an antigen-specific T cell, comprising a step of contacting lymphocytes with the peptide according to claim 1.

12. A method for preparing an antigen-presenting cell, comprising a step of contacting a cell having an antigen-presenting ability with the peptide according to claim 1.

13. The method according to claim 12, wherein contacting the cell having an antigen-presenting ability with the peptide is performed by:
a step of culturing the cell with the peptide, and binding and presenting the peptide to an HLA molecule of the cell, or
a step of introducing a vector capable of expressing the peptide into the cell to express the peptide.

14. The method according to claim 12, wherein the cell having an antigen-presenting ability is a dendritic cell.

15. A gene encoding a T cell receptor (TCR) reactive to the peptide according to claim 1 isolated from an antigen-specific T cell clone for the peptide.

16. The gene according to claim 15, wherein the gene encoding a TCR is a gene encoding a TCRα chain or a gene encoding a TCRf3 chain, or both thereof.

17. The gene according to claim 15, wherein the gene is the full length or a part of a complementarity determining region (CDR) gene.

18. AT cell having a modified TCR gene (TCR-T cell), created by introducing the gene according to claim 15 into a T cell.


SEQUENCE LISTING (32 sequences)

SEQ ID NO: 1 - protein, 27 aa, Homo sapiens
MTEYKLVVVGADGVGKSALTIQLIQNH

SEQ ID NO: 2 - protein, 27 aa, Homo sapiens
MTEYKLVVVGAVGVGKSALTIQLIQNH

SEQ ID NO: 3 - protein, 27 aa, Homo sapiens
MTEYKLVVVGACGVGKSALTIQLIQNH

SEQ ID NO: 4 - protein, 27 aa, Homo sapiens
MTEYKLVVVGARGVGKSALTIQLIQNH

SEQ ID NO: 5 - protein, 27 aa, Homo sapiens
MTEYKLVVVGAGDVGKSALTIQLIQNH

SEQ ID NO: 6 - protein, 27 aa, Homo sapiens
GETCLLDILDTAGKEEYSAMRDQYMRT

SEQ ID NO: 7 - protein, 27 aa, Homo sapiens
GETCLLDILDTAGREEYSAMRDQYMRT

SEQ ID NO: 8 - protein, 27 aa, Homo sapiens
EALEYFMKQMNDARHGGWTTKMDWIFH

SEQ ID NO: 9 - protein, 27 aa, Homo sapiens
KAISTRDPLSEITKQEKDFLWSHRHYC

SEQ ID NO: 10 - protein, 27 aa, Homo sapiens
GRITKICDFGLARVIKNDSNYVVKGNA

SEQ ID NO: 11 - protein, 15 aa, Homo sapiens
EALEYFMKQMNDARH

SEQ ID NO: 12 - protein, 15 aa, Homo sapiens
ALEYFMKQMNDARHG

SEQ ID NO: 13 - protein, 15 aa, Homo sapiens
LEYFMKQMNDARHGG

SEQ ID NO: 14 - protein, 15 aa, Homo sapiens
EYFMKQMNDARHGGW

SEQ ID NO: 15 - protein, 14 aa, Homo sapiens
YFMKQMNDARHGGW

SEQ ID NO: 16 - protein, 14 aa, Homo sapiens
FMKQMNDARHGGWT

SEQ ID NO: 17 - protein, 14 aa, Homo sapiens
MKQMNDARHGGWTT

SEQ ID NO: 18 - protein, 14 aa, Homo sapiens
KQMNDARHGGWTTK

SEQ ID NO: 19 - protein, 13 aa, Homo sapiens
QMNDARHGGWTTK

SEQ ID NO: 20 - protein, 13 aa, Homo sapiens
MNDARHGGWTTKM

SEQ ID NO: 21 - protein, 13 aa, Homo sapiens
NDARHGGWTTKMD

SEQ ID NO: 22 - protein, 13 aa, Homo sapiens
DARHGGWTTKMDW

SEQ ID NO: 23 - protein, 13 aa, Homo sapiens
ARHGGWTTKMDWI

SEQ ID NO: 24 - protein, 15 aa, Homo sapiens
FGLARVIKNDSNYVV

SEQ ID NO: 25 - protein, 15 aa, Homo sapiens
GLARVIKNDSNYVVK

SEQ ID NO: 26 - protein, 15 aa, Homo sapiens
LARVIKNDSNYVVKG

SEQ ID NO: 27 - protein, 15 aa, Homo sapiens
ARVIKNDSNYVVKGN

SEQ ID NO: 28 - protein, 15 aa, Homo sapiens
RVIKNDSNYVVKGNA

SEQ ID NO: 29 - protein, 112 aa, Homo sapiens
QQVKQSPQSLIVQKGGISIINCAYENTAFEYFPWYQQFPGKGPALLIAIRPDVSEKKEGRFTISFNKSAKQFSLHIMDSQPGDSATYFCAASGSYNNNDMRFGAGTRLTVKP

SEQ ID NO: 30 - protein, 117 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYASPGTGYSGELFFGEGSRLTVL

SEQ ID NO: 31 - protein, 110 aa, Homo sapiens
GQSLEQPSEVTAVEGAIVQINCTYQTSGFYGLSWYQQHDGGAPTFLSYNALDGLEETGRFSSFLSRSDSYGYLLLQELQMKDSASYFCAVRDNAGNMLTFGGGTRLMVKP

SEQ ID NO: 32 - protein, 113 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIPNLGYGYTFGSGTRLTVV